(19) United States
(12) Patent Application Publication
(10) Pub. No.: US 2011/0297044 A1
(43) Pub. Date: Dec. 8, 2011

(54) OLIGOANILINE EXFOLIATING AGENTS, EXFOLIATED PLATELET-SHAPED CLAY COMPRISING THE EXFOLIATING AGENT AND PREPARATION THEREOF

(76) Inventors: Tsung-Yen TSAI, Taoyuan County (TW); Chun-Yi Lee, Taoyuan County (TW)

(21) Appl. No.: 12/951,668

(22) Filed: Nov. 22, 2010

(30) Foreign Application Priority Data
Jun. 3, 2010 (TW) 99117897

(51) Int. Cl. C04B 14/00; C07C 211/54

(52) U.S. Cl. 106/416; 564/434

(57) ABSTRACT

In an embodiment, an oligoaniline exfoliating agent, as shown in Formula (I), is provided.

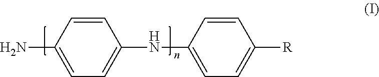

In Formula (I), R is —H or —NH₂, and n is 3 to 30. In another embodiment, exfoliated platelet-shaped clay including the oligoaniline exfoliating agent and a method for preparing the exfoliated platelet-shaped clay are provided.

### CROSS REFERENCE TO RELATED APPLICATIONS

This Application claims priority of Taiwan Patent Application No. 099117897, filed on Jun. 3, 2010, the entirety of which is incorporated by reference herein.

### BACKGROUND OF THE INVENTION

1. Field of the Invention

The invention relates to an exfoliating agent, and more particularly to an oligoaniline exfoliating agent, exfoliated platelet-shaped clay comprising the exfoliating agent and preparation thereof.

2. Description of the Related Art

For a conventional technique, a surfactant is utilized as a modifier to enter inorganic layered-structure clay through ion-exchange and further blend with polymers. The inorganic layered-structure clay is unfolded by the modifier. A monomer then enters the unfolded space and is polymerized to form a polymer to destroy the layered structure of the inorganic clay to form exfoliated platelet-shaped inorganic clay dispersed in polymer substrate.

### BRIEF SUMMARY OF THE INVENTION

One embodiment of the invention provides an oligoaniline exfoliating agent represented by Formula (I):

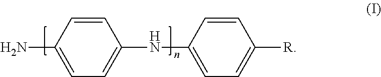

In Formula (I), R is —H or —NH₂, and n is 3 to 30.

One embodiment of the invention provides an exfoliated platelet-shaped clay, comprising an exfoliated platelet-shaped clay, and an oligoaniline located between the exfoliated platelet-shaped clay, represented by Formula (I):

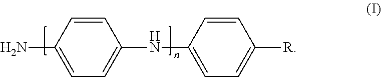

In Formula (I), R is —H or —NH₂, and n is 3 to 30.

One embodiment of the invention provides a method for preparing an exfoliated platelet-shaped clay, comprising blending an oligoaniline and an inorganic acid to form a first solution, swelling a clay in a liquid to form a second solution, blending the first and second solutions to form a blending solution, washing the blending solution with a solvent, separating the blending solution to collect a solid, and drying the solid to form an exfoliated platelet-shaped clay, wherein the oligoaniline is represented by Formula (I):

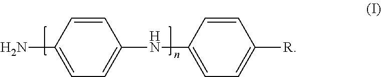

In Formula (I), R is —H or —NH₂, and n is 3 to 30.

The invention provides an oligoaniline as an exfoliating agent for layered clay. In the oligoaniline structure, its amino group (—NH₂) is reacted with an inorganic acid to form —NH₃⁺ (protonation). The charged functional group —NH₃⁺ is further ion-exchanged with the electric charges formed between the layered clay to facilitate the insertion of the oligoaniline between the layered clay. The ionic bond and Van der Waals force formed in the original layered structure are forcedly broken due to the steric hindrance formed by the oligoaniline such that the spacing of the layered clay is unfolded to finally form a disorder exfoliated platelet-shaped clay structure. Compared to the conventional technique requiring durable polymerization, which is polymerized from an inserted initial monomer to a polymer to exfoliate clay, to be performed, the invention utilizing the oligoaniline exfoliating agent substantially reduces preparation time. The invention intercalates the oligoaniline into swelled layered clay to exfoliate clay with simple physical agitation. Therefore, the invention is a novel method to prepare the exfoliated platelet-shaped clay.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 illustrates exfoliation effects of exfoliating agent solutions with various pH values on inorganic clay according to one embodiment of the invention;

FIG. 2 illustrates exfoliation effects of unacidification and acidification of exfoliating agents on inorganic clay according to one embodiment of the invention;

FIG. 3 illustrates an exfoliated composite material formed by adding exfoliated inorganic clay to a PMMA wetting preparation according to one embodiment of the invention;

FIG. 4 illustrates an exfoliated composite material formed by adding exfoliated inorganic clay to epoxy in-situ polymerization according to one embodiment of the invention;

FIG. 5 illustrates an exfoliated composite material formed by adding exfoliated inorganic clay to a PLA twin-screw dry preparation according to one embodiment of the invention;

FIG. 6 illustrates an exfoliated composite material formed by adding exfoliated inorganic clay to polymers of Examples 4 to 6 according to one embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

One embodiment of the invention provides an oligoaniline exfoliating agent represented by Formula (I):

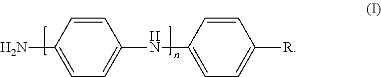

In Formula (I), R may be —H or —NH₂, and n may be 3 to 30. The structure of the disclosed oligoaniline exfoliating agent may comprise —NH₂ functional groups or other functional groups capable of performing ion-exchange with the electric charges formed between layered clay.

The molecular weight of the disclosed oligoaniline exfoliating agent is less than 10,000, preferably less than 3,000.

One embodiment of the invention provides an exfoliated platelet-shaped clay, comprising an exfoliated platelet-shaped clay, and an oligoaniline located between the exfoliated platelet-shaped clay, represented by Formula (I):

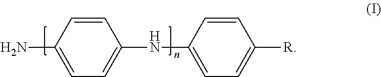

In Formula (I), R may be —H or —NH₂, and n may be 3 to 30. The structure of the disclosed oligoaniline exfoliating agent may comprise —NH₂ functional groups or other functional groups capable of performing ion-exchange with the electric charges formed between layered clay.

The disclosed exfoliated platelet-shaped clay may comprise smectite clay, vermiculite, halloysite, sericite, mica or layered double hydroxides (LDHs). The smectite clay may comprise montmorillonite, saponite, beidellite, nontronite or hectorite.

The molecular weight of the disclosed oligoaniline exfoliating agent is less than 10,000, preferably less than 3,000.

The disclosed exfoliated platelet-shaped clay has a d-spacing exceeding 27 Å.

One embodiment of the invention provides a method for preparing the exfoliated platelet-shaped clay, comprising the following steps. First, an oligoaniline and an inorganic acid are blended to form a first solution. Meanwhile, clay is swelled in a liquid to form a second solution. Next, the first and second solutions are blended to form a blending solution. The blending solution represents then washed with a solvent. Next, the blending solution is separated to collect a solid. The solid represents then dried to form the exfoliated platelet-shaped clay. The oligoaniline is represented by Formula (I):

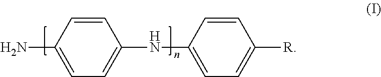

In Formula (I), R may be —H or —NH₂, and n may be 3 to 30. The structure of the disclosed oligoaniline exfoliating agent may comprise —NH₂ functional groups or other functional groups capable of performing ion-exchange with the electric charges formed between layered clay.

The molecular weight of the oligoaniline is less than 10,000, preferably less than 3,000. The inorganic acid may comprise nitric acid, hydrochloric acid, sulfuric acid, phosphoric acid or the like. The first solution is completely acidified, for example, adjusting its pH to less than 3.

The clay may comprise smectite clay, vermiculite, halloysite, sericite, mica or layered double hydroxides (LDHs). The smectite clay may comprise montmorillonite, saponite, beidellite, nontronite or hectorite. In one embodiment, the disclosed clay has a cation exchange capacity (CEC) of about 50-200 meq/100 g. The liquid used to swell the clay may comprise deionized water, distilled water, ethanol or the like. In the second solution, the clay and the liquid have a weight ratio of about 1:25 to 1:50.

In the blending solution, the oligoaniline has a weight of at least 1 or 1.2 times that of the cation exchange capacity (CEC) of the clay. The first and second solutions are blended with physical agitation, for example, magnetic agitation, ultrasonic shaking, sand mill agitation, drum agitation, ball mill agitation, rocking and shaking, mechanical agitation or the like. In the invention, the physical agitation used to blend the first and second solutions facilitates intercalation of the oligoaniline into the swelled layered clay.

The solvent used to wash the blending solution may be selected from a solvent capable of dissolving the oligoaniline; for example, alkane, alcohol, ether or the like.

The blending solution is separated by, for example, solid-liquid separation methods such as centrifugation, concentration/extraction, heating, vacuum drying, freeze drying, and supercritical fluid separation methods or the like. For example, the unintercalated oligoaniline is removed by centrifugation for several times.

The solid is dried by, for example, heating, concentration/extraction, freeze drying or the like. The residual solvent after washing the blending solution is removed during this step.

The prepared exfoliated platelet-shaped clay has a d-spacing exceeding 27 Å.

The invention provides an oligoaniline as an exfoliating agent for layered clay. In the oligoaniline structure, its amino group (—NH₂) is reacted with an inorganic acid to form —NH₃⁺ (by protonation). The charged functional group —NH₃⁺ is further ion-exchanged with the electric charges formed between the layered clay to facilitate the insertion of the oligoaniline between the layered clay. The ionic bond and van der Waals force formed in the original layered structure are forcedly broken due to the steric hindrance formed by the oligoaniline such that the spacing of the layered clay is unfolded to finally form a disorder exfoliated platelet-shaped clay structure. Compared to the conventional technique requiring durable polymerization, which is polymerized from an inserted initial monomer to a polymer to exfoliate clay, to be performed, the invention utilizing the oligoaniline exfoliating agent substantially reduces preparation time. The invention intercalates the oligoaniline into swelled layered clay to exfoliate clay with simple physical agitation. Therefore, the invention is a novel method to prepare the exfoliated platelet-shaped clay.

Additionally, the disclosed disorder exfoliated nano-level silicon oxide layer inorganic platelet-shaped clay acts a substrate and is further blended with a polymer which is polymerized from a monomer to form an exfoliated organic/inorganic hybrid composite material. The flame retardancy, gas barrier capability, thermal property and mechanical property of the composite material are effectively improved due to blending with the exfoliated platelet-shaped clay. Additionally, the two extremities of the disclosed oligoaniline are designed to respectively connect an amino group (—NH₂) and blend with an aniline monomer and an oxidant to form a conductive polymer, which may be further applied in, for example, anti-static materials, anti-corrosion paints, gas detectors, electrochromic display materials, photodiode materials, electromagnetic wave shielding bodies or biomedical sensors.

### Example 1

### Preparation of Exfoliated Platelet-Shaped Clay

0.3 g of montmorillonite (CEC: 168 meq/100 g) was swelled in 5 ml of deionized water to form a solution A. 0.28 g (1.2 times that of the CEC of the montmorillonite) of pentaaniline (Mw: 455) was added to 4 ml of deionized water (pH 2 to 4, adjusted by 0.001M H₂SO₄) to form a solution B. After solution A and solution B were respectively agitated with mechanical agitation (450 rpm) under room temperature for 24 hrs, solution A and solution B were blended to form a solution C. Solution C was continuously agitated with mechanical agitation (600 rpm) under room temperature for 24 hrs to exchange the aniline pentamer of the quaternary ammonium salt (RNH₃⁺) with the sodium ions in the montmorillonite to facilitate insertion of the pentaaniline between the montmorillonite to exfoliate the layered structure of the montmorillonite to form exfoliated platelet-shaped montmorillonite.

Solution C was washed with ethanol under high-speed centrifugation (3,000 rpm) for several times to remove unreacted pentaaniline. The precipitates were then vacuum freeze dried (−20° C., ˜0.005 torr) to remove residual ethanol to form an exfoliated platelet-shaped montmorillonite mass. Exfoliated platelet-shaped montmorillonite powder was obtained after the montmorillonite mass was crushed by a physical mill.

### Example 2

### Exfoliation Effects of Exfoliating Agent Solutions with Various pH Values on Inorganic Clay

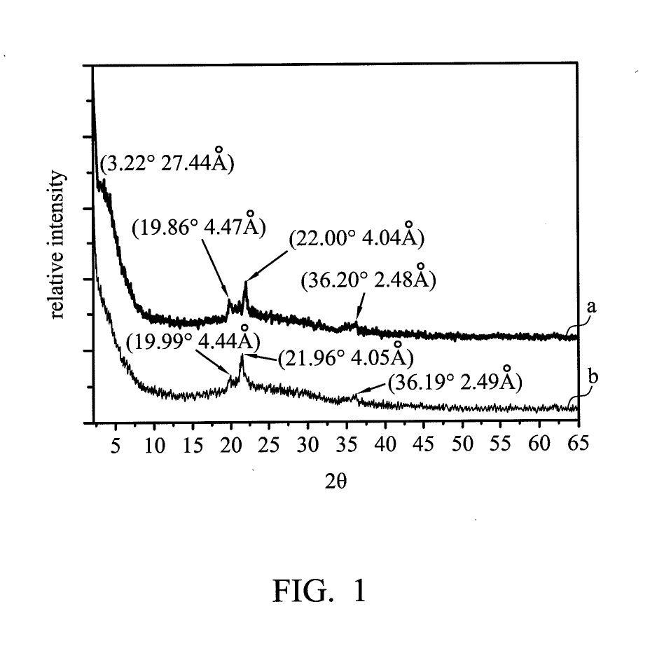

Referring to FIG. 1, exfoliation effects of exfoliating agent solutions with various pH values on inorganic clay are illustrated. FIG. 1 shows X-ray diffraction of clay powder. In the figure, the curve a represents the exfoliation effect of a pentaaniline exfoliating agent solution with pH 3.70 on montmorillonite (NTC-C34). Curve b represents the exfoliation effect of a pentaaniline exfoliating agent solution with pH 2.57 on montmorillonite (NTC-C34). The results indicate that when the pH of the pentaaniline exfoliating agent solution was 3.70, the (001) characteristic peak of the montmorillonite (NTC-C34) was exhibited at 2θ=3.22° (d-spacing=27.44 Å). However, when the pH of the pentaaniline exfoliating agent solution was lowered to 2.57, the (001) characteristic peak of the montmorillonite (NTC-C34) was left shifted into the background. The inorganic clay was completely exfoliated when no (001) characteristic peak was exhibited within the range from 0.00° to 7.00°. Thus, in accordance with FIG. 1, the montmorillonite (NTC-C34) achieved partial exfoliation (a (001) characteristic peak was exhibited at 2θ=3.22° by blending with the pentaaniline exfoliating agent solution with pH 3.70. However, the montmorillonite (NTC-C34) achieved complete exfoliation by blending with the pentaaniline exfoliating agent solution with pH 2.57.

### Example 3

### Exfoliation Effects of Acidification of Exfoliating Agents on Inorganic Clay

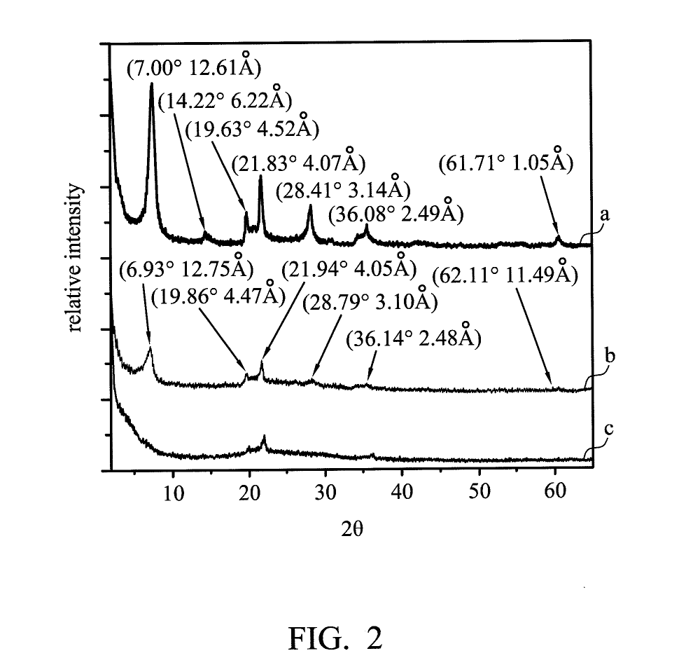

Referring to FIG. 2, exfoliation effects of unacidification and acidification of exfoliating agents on inorganic clay are illustrated. FIG. 2 shows X-ray diffraction of clay powder. In the figure, the curve a represents the pure montmorillonite (NTC-C34) without addition of any exfoliating agents. The curve b represents the exfoliation effect of an unacidified pentaaniline exfoliating agent on montmorillonite (NTC-C34). The curve c represents the exfoliation effect of an acidified pentaaniline exfoliating agent on montmorillonite (NTC-C34). The results indicate that when no exfoliating agent was added, the (001) characteristic peak of the montmorillonite (NTC-C34) was exhibited at 2θ=7.00° (d-spacing=12.61 Å). When the unacidified pentaaniline exfoliating agent was added, the (001) characteristic peak of the montmorillonite (NTC-C34) was exhibited at 2θ=6.93° (d-spacing=12.75 Å). However, when the acidified pentaaniline exfoliating agent was added, the (001) characteristic peak of the montmorillonite (NTC-C34) was left shifted into the background. When the unacidified pentaaniline exfoliating agent was added, due to no ion-exchange between the unacidified pentaaniline exfoliating agent and the inorganic clay, the intercalation effect on the montmorillonite (NTC-C34) was omitted and its (001) characteristic peak was similar to that of the pure montmorillonite (NTC-C34). However, when the acidified pentaaniline exfoliating agent was added, the montmorillonite (NTC-C34) was completely exfoliated. Thus, the acidification of exfoliating agents is a critical factor for intercalation and clay exfoliation.

### Example 4

### Preparation of Exfoliated Platelet-Shaped Montmorillonite/PMMA Composite Material

0.485 g of poly(methylmethacrylate) (PMMA, Aldrich, Mw: 350,000) was dissolved in 5 g of 1-methyl-2-pyrrolidnone (NMP, TEDIA, 99.5%) with mechanical agitation (450 rpm) under room temperature until PMMA was completely dissolved to form a solution A1. 0.015 g of exfoliated platelet-shaped montmorillonite powder (as Example 2 curve b, 3 wt %) was dissolved in 4.5 g of NMP with mechanical agitation (450 rpm) under room temperature until the montmorillonite was completely dispersed to form a solution B1. Solution A1 and solution B1 were uniformly blended with an agitation (600 rpm) under room temperature to form a solution C1. 4 ml of solution C1 was drawn by a 5 ml syringe and uniformly coated on a 6×6 cm glass plate. After NMP was slowly evaporated at 60° C. in hood, PMMA and the exfoliated platelet-shaped montmorillonite powder in the solution was formed into a composite material film.

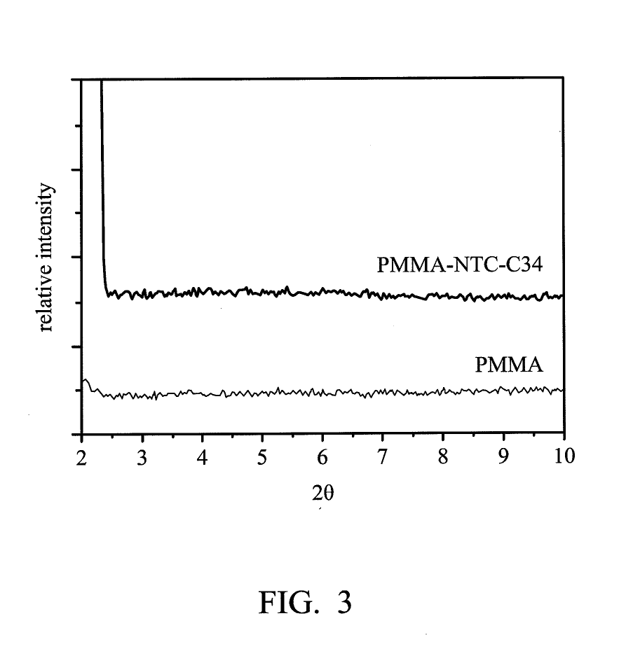

The dispersivity of the composite material is illustrated in FIG. 3. In accordance with the XRD test results, the (001) characteristic peak of the PMMA-NTC-C34 was omitted within the range from 2° to 10° (2θ). The results indicate that the exfoliated platelet-shaped montmorillonite was uniformly dispersed in the thermoplastic polymer. The addition, the exfoliated platelet-shaped montmorillonite can be combined by PMMA wetting preparation.

### Example 5

### Preparation of Exfoliated Platelet-Shaped Montmorillonite/Epoxy Composite Material

0.15 g of exfoliated platelet-shaped montmorillonite powder (as Example 2 curve b, 3 wt %) was added to 3 g of propylene glycol mono-methyl ether and uniformly stirred to form a solution. 3.4643 g of a hardener was then added to the solution and stirred under room temperature for one day. Next, 5.2343 g of a novolac epoxy resin was added to the solution and stirred under room temperature for one day. The resulting solution was then poured into a mold. After the solvent was removed in a vacuum oven (190° C., ˜0.05 torr), the mold was placed in an oven conducted with hot air (190° C.) for 3 hrs until the novolac epoxy resin mass was hardened.

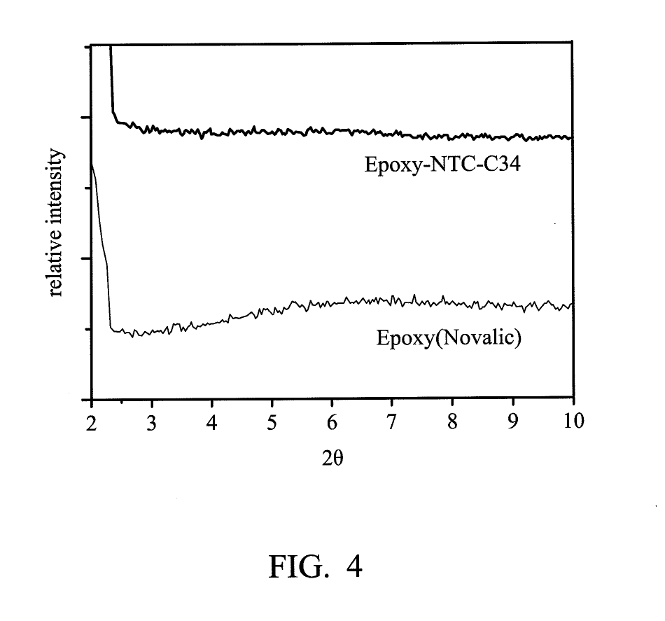

The dispersivity of the composite material is illustrated in FIG. 4. In accordance with the XRD test results, the (001) characteristic peak of the Epoxy-NTC-C34 was omitted within the range from 2° to 10° (2θ). The conjugate structure of the pentaaniline facilitated the novolac epoxy resin when performing ring-opened in-situ polymerization such that the exfoliated platelet-shaped montmorillonite was dispersed in the novolac epoxy resin to form an exfoliated composite material.

### Example 6

### Preparation of Exfoliated Platelet-Shaped Montmorillonite/PLA Composite Material

5 g of polylactide (PLA, Nature Green™ 4032D, Mw: 180,000 to 200,000) was added to a twin-screw extruder (DSM Xplore 15 ml Microcompounder) and melted and stirred at 180° C. with 50 rpm. 0.1 g of exfoliated platelet-shaped montmorillonite powder (as Example 2 curve b, 2 phr) was added to the twin-screw extruder. After blending for 5 mins, a composite material of PLA and the exfoliated platelet-shaped montmorillonite was extruded and cut into a proper composite material particles.

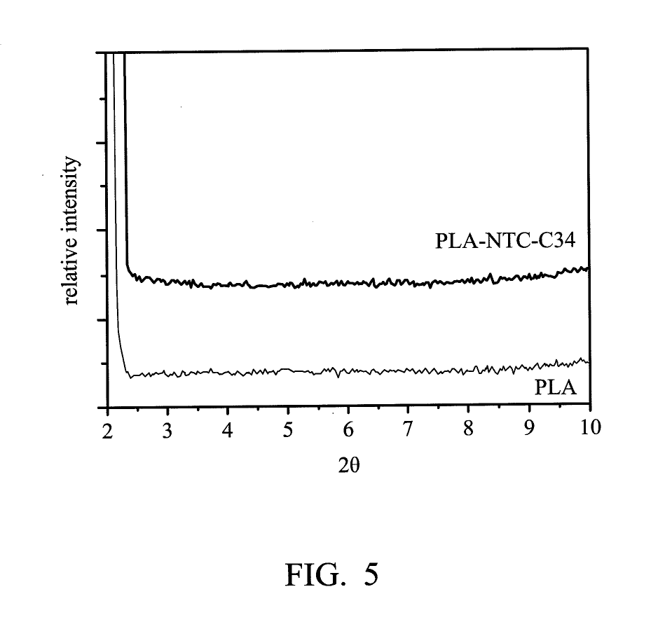

The composite material particles were then hot pressed into a film by a hot press (upper plate: 200° C., lower plate: 200° C., pressure: 700 psi). The dispersivity of the composite material film is illustrated in FIG. 5. In accordance with the XRD test results, the (001) characteristic peak of the PLA-NTC-C34 was omitted within the range from 2° to 10° (2θ). The results indicate that the exfoliated platelet-shaped montmorillonite powder was dispersed in PLA through the twin screw shear force to form an exfoliated nano composite material.

The thermal property of the composite material film is shown in Table 1. The thermal degradation temperature thereof was measured by TGA. T_{5d} represents a corresponding temperature when 5% of a total weight was lost. T_{10d} represents a corresponding temperature when 10% of a total weight was lost. The thermal degradation temperature of the composite material film, which was added with the exfoliated platelet-shaped montmorillonite, improved.

TABLE 1

| Sample | T_{5d} (° C.) | T_{10d} (° C.) |
|---|---|---|
| PLA | 315.4 | 321.6 |
| PLA + montmorillonite | 319.9 | 328.5 |

### Example 7

The exfoliated platelet-shaped montmorillonite powder (as Example 2 curve b) was respectively added to the polymers of Examples 4 to 6 to form composite materials.

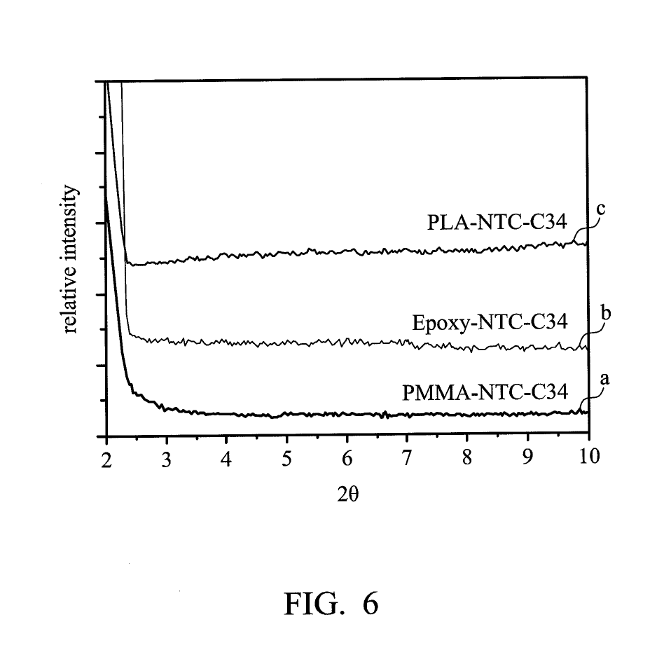

The dispersivity of the composite materials is illustrated in FIG. 6 (XRD test). In the figure, a represents the composite material film of PMMA and the exfoliated platelet-shaped montmorillonite powder (PMMA-NTC-C34), b represents the composite material mass of epoxy and the exfoliated platelet-shaped montmorillonite powder (Epoxy-NTC-C34), and c represents the composite material film of PLA and the exfoliated platelet-shaped montmorillonite powder (PLA-NTC-C34). The (001) characteristic peaks of the three composite materials were omitted within the range from 2° to 10° (2θ). The results indicate that the layered montmorillonite was uniformly dispersed in the polymers. When the ionic bond and van der Waals force formed in the platelet-shaped structure were broken, a platelet-shaped structure was uniformly dispersed in the polymers. The pentaaniline located between the platelet-shaped structures prevented the disorder platelet-shaped structures from returning to order. Additionally, partially exfoliated montmorillonite (unlike Example 2 curve b) can also be further dispersed using solvent to form an exfoliated composite material.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An oligoaniline exfoliating agent, represented by Formula (I): 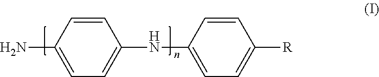
wherein
R is —H or —NH₂; and
n is 3 to 30.

2. The oligoaniline exfoliating agent as claimed in claim 1, wherein the oligoaniline exfoliating agent has a molecular weight of less than 10,000.

3. The oligoaniline exfoliating agent as claimed in claim 1, wherein the oligoaniline exfoliating agent has a molecular weight of less than 3,000.

4. An exfoliated platelet-shaped clay, comprising:
an exfoliated platelet-shaped clay; and
an oligoaniline located between the exfoliated platelet-shaped clay, represented by Formula (I): 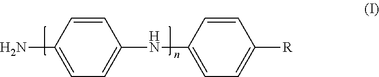
wherein
R is —H or —NH₂; and
n is 3 to 30.

5. The exfoliated platelet-shaped clay as claimed in claim 4, wherein the exfoliated platelet-shaped clay comprises smectite clay, vermiculite, halloysite, sericite, mica or layered double hydroxides (LDHs).

6. The exfoliated platelet-shaped clay as claimed in claim 5, wherein the smectite clay comprises montmorillonite, saponite, beidellite, nontronite or hectorite.

7. The exfoliated platelet-shaped clay as claimed in claim 4, wherein the oligoaniline has a molecular weight of less than 10,000.

8. The exfoliated platelet-shaped clay as claimed in claim 4, wherein the oligoaniline has a molecular weight of less than 3,000.

9. The exfoliated platelet-shaped clay as claimed in claim 4, wherein the exfoliated platelet-shaped clay has a d-spacing exceeding 27 Å.

10. A method for preparing an exfoliated platelet-shaped clay, comprising:
blending an oligoaniline and an inorganic acid to form a first solution, wherein the oligoaniline is represented by Formula (I): 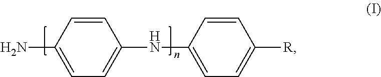
wherein R is —H
or —NH₂, and n is 3 to 30;
swelling a clay in a liquid to form a second solution;
blending the first and second solutions to form a blending solution;
washing the blending solution with a solvent;
separating the blending solution to collect a solid; and
drying the solid to form an exfoliated platelet-shaped clay.

11. The method for preparing the exfoliated platelet-shaped clay as claimed in claim 10, wherein the inorganic acid comprises nitric acid, hydrochloric acid, sulfuric acid, phosphoric acid or the like.

12. The method for preparing the exfoliated platelet-shaped clay as claimed in claim 10, wherein the first solution has a pH less than 3.

13. The method for preparing the exfoliated platelet-shaped clay as claimed in claim 10, wherein the clay comprises montmorillonite, saponite, beidellite, nontronite or hectorite.

14. The method for preparing the exfoliated platelet-shaped clay as claimed in claim 10, wherein the clay has a cation exchange capacity (CEC) of about 50-200 meq/100 g.

15. The method for preparing the exfoliated platelet-shaped clay as claimed in claim 14, wherein the oligoaniline has a weight of at least 1.2 times that of the cation exchange capacity (CEC) of the clay.

16. The method for preparing the exfoliated platelet-shaped clay as claimed in claim 10, wherein the first and second solutions are blended with a physical agitation.
